(19) United States
(12) Patent Application Publication
(10) Pub. No.: US 2025/0331816 A1
(43) Pub. Date: Oct. 30, 2025

(54) ULTRASOUND DIAGNOSTIC APPARATUS

(71) Applicant: Konica Minolta, Inc., Tokyo (JP)

(72) Inventors: Takahiko SHIRAISHI, Tokyo (JP); Tatsushi CHIHARA, Tokyo (JP)

(21) Appl. No.: 19/175,022

(22) Filed: Apr. 10, 2025

(30) Foreign Application Priority Data
Apr. 24, 2024 (JP) 2024-070891

(51) Int. Cl. A61B 8/00; A61B 50/13

(52) U.S. Cl. CPC A61B 8/4405; A61B 50/13

(57) ABSTRACT

An ultrasound diagnostic apparatus includes a cart and an apparatus main body. The ultrasound diagnostic apparatus is configured to be movable in a state where the apparatus main body is mounted on the cart. The cart includes a support pillar part and a plate-shaped table part that protrudes from the support pillar part in a substantially horizontal direction. On a lower surface side of the table part, a housing tray is attached so as to be freely opened and closed with respect to the table part. The housing tray is capable of housing at least an extra-length cable of a peripheral device.

### CROSS-REFERENCE TO RELATED APPLICATIONS

The entire disclosure of Japanese Patent Application No.2024-070891, filed on Apr. 24, 2024, including description, claims, drawings and abstract is incorporated herein by reference in its entity.

### TECHNICAL FIELD

The present invention relates to an ultrasound diagnostic apparatus.

### DESCRIPTION OF RELATED ART

According to a known ultrasound diagnostic apparatus, the main body of the ultrasound diagnostic apparatus is mounted on a cart. Such an ultrasound diagnostic apparatus is used while being moved between rooms in a facility.

For example, Japanese Unexamined Patent Publication No. 2011-36302 discloses an ultrasound diagnostic apparatus including a housing section that houses a probe cable that connects an ultrasound probe (probe) and the main body of the ultrasound diagnostic apparatus.

However, not only an ultrasound probe (probe) but also various peripheral devices may be connected to an ultrasound diagnostic apparatus and used together with the ultrasound diagnostic apparatus. In such a case, the ultrasound diagnostic apparatus is moved while being connected to many peripheral device cables.

When the ultrasound diagnostic apparatus is moved, cables of the peripheral devices may be caught in various places in the facility, and connectors of the peripheral devices may be damaged. Furthermore, the cables of the peripheral devices may be brought into contact to or caught by other devices in the facility, thereby damaging the other devices.

The present invention has been made in consideration of the above problems. An object of the present invention is to prevent a cable of a peripheral device from being caught by other devices and so forth in a facility and to reduce the risk of damaging the peripheral device and the other devices when the ultrasound diagnostic apparatus is moved.

### SUMMARY OF THE INVENTION

In order to solve the above problem, according to an aspect of the present invention, there is provided an ultrasound diagnostic apparatus including: a cart; and an apparatus main body to be mounted on the cart, wherein: the ultrasound diagnostic apparatus is configured to be movable in a state where the apparatus main body is mounted on the cart, the cart includes a support pillar part and a plate-shaped table part that protrudes from the support pillar part in a substantially horizontal direction, and on a lower surface side of the table part, a housing tray is attached so as to be freely opened and closed with respect to the table part, the housing tray being capable of housing at least an extra-length cable of a peripheral device.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein

FIG. 1 is a perspective external view of an ultrasound diagnostic apparatus;

FIG. 2 is a perspective view of main components of the ultrasound diagnostic apparatus as viewed from the lower side of a table part;

FIG. 3 is a perspective view of main components of the ultrasound diagnostic apparatus illustrating the inside of a housing space with a housing tray open;

FIG. 4 is a perspective view of the housing tray that is removed from the ultrasound diagnostic apparatus;

FIG. 5 is a perspective view of the inside of the housing space in FIG. 3 in which an extra-length cable and a peripheral device are housed;

FIG. 6 is a perspective view of main parts of the table part and its surroundings as viewed from the back;

FIG. 7 is a perspective view of a support part in FIG. 6 as viewed from the lateral side, wherein a camera attachment unit is attached to the support part;

FIG. 8 is a perspective view of main components of the ultrasound diagnostic apparatus as viewed from diagonally behind, wherein the housing tray is open and the housing space houses the extra-length cable and the peripheral device;

FIG. 9 is a perspective view of main parts as viewed from diagonally behind, wherein the housing tray of FIG. 8 is closed; and

FIG. 10 is a perspective view of main parts illustrating a state where an extra probe is housed in the housing space illustrated in FIG. 3.

### DETAILED DESCRIPTION

Hereinafter, an embodiment of the present invention will be described with reference to the drawings. However, the technical scope of the present invention is not limited to the following embodiments and illustrated examples.

### Configuration of Ultrasound Diagnostic Apparatus

First, a configuration of an ultrasound diagnostic apparatus 1 according to the present embodiment will be described.

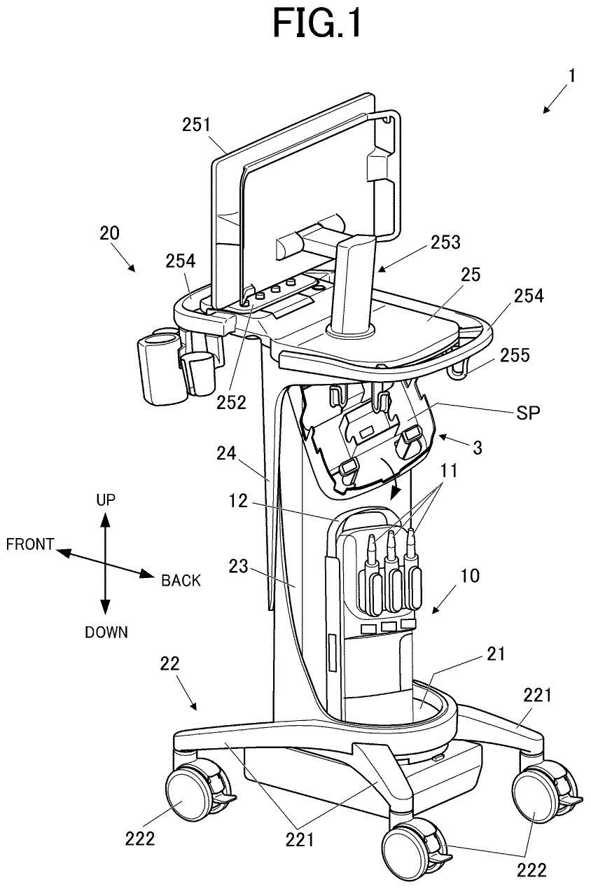

The ultrasound diagnostic apparatus 1 is installed in an examination room and so forth of a medical facility, such as a hospital. A user of the ultrasound diagnostic apparatus 1, such as a physician or a technician, places an ultrasound probe (probe, not illustrated in FIG. 1 and so forth) that transmits and receives ultrasound waves on a subject (patient). The ultrasound diagnostic apparatus 1 is an apparatus that transmits and receives ultrasound waves from an ultrasound probe and generates an ultrasound image from echo signals corresponding to echoes from a subject. The ultrasound diagnostic apparatus 1 of the present embodiment is configured to be movable in the medical facility, such as a hospital.

FIG. 1 is a perspective external view of the ultrasound diagnostic apparatus 1. Hereinafter, up, down, front, and back refer to the directions illustrated in FIG. 1.

As illustrated in FIG. 1, the ultrasound diagnostic apparatus 1 includes an ultrasound diagnostic apparatus main body (hereinafter referred to as an apparatus main body) 10 and a cart 20 (dolly). The ultrasound diagnostic apparatus 1 is configured to be movable with the apparatus main body 10 mounted on the cart (dolly) 20. Hereinafter, the apparatus main body 10 and the cart (dolly) 20 will be described in detail.

### Configuration of Apparatus Main Body

The apparatus main body 10 transmits drive signals to the ultrasound probe, receives echo signals as electrical signals from the ultrasound probe, and generates ultrasound image data, based on the echo signals. The apparatus main body 10 can be removed from the cart 20 and carried. The ultrasound probe is attachable to and detachable from the apparatus main body 10.

On the front surface of the apparatus main body 10 that is exposed to the front when the apparatus main body 10 is mounted on the cart (dolly) 20, three connectors 11 for connecting ultrasound probes are arranged side by side. Although not shown, a connector and so forth for connecting a plug at the end of a power cable is provided on the front surface and so forth of the apparatus main body 10.

The apparatus main body 10 also includes, on its upper surface, a grip part 12 that is used when the apparatus main body 10 is attached to or detached from the cart 20 or when the apparatus main body 10 is carried.

### Configuration of Cart

Next, the configuration of the cart 20 will be described. The cart 20 is a movable cart that displays ultrasound image data generated by the apparatus main body 10. The apparatus main body 10 is detachably mounted on the cart 20.

The cart 20 includes a main body mounting part 21 and a caster part 22. Furthermore, as illustrated in FIG. 1, the cart 20 of the present embodiment includes at least a support column part 23 and a table part 25.

As illustrated in FIG. 1, the main body mounting part 21 is a pedestal for mounting the apparatus main body 10. The main body mounting part 21 is shaped such that the main body mounting part 21 can support the bottom surface of the apparatus main body 10 when the apparatus main body 10 is attached. Although not illustrated, the main body mounting part 21 includes connection terminals to be connected to connection terminals (not illustrated) of the apparatus main body 10 at positions corresponding to the respective connection terminals.

As shown in FIG. 1, the caster part 22 includes four legs 221 that radially extend from the main body mounting part 21 and casters 222 provided at the ends of the respective legs 221. With the rotation and turning of the casters 222 of the caster part 22, the cart 20 and the apparatus main body 10 attached to the cart 20 are movable in the front, back, left, and right directions.

As shown in FIG. 1 and so forth, the support column part 23 is erected on the end of the front side of the main body mounting part 21. The support column part 23 supports the apparatus main body 10 mounted on the main body mounting part 21 from the back surface side (the side on which the connectors 11 are not provided). A holding frame 24 is attached to the support column part 23. The table part 25 is held on the upper end side of the holding frame 24. The holding frame 24 and the table part 25 held by the holding frame 24 are slidable in the up-down direction along the support column part 23.

The table part 25 has a substantially flat plate shape and projects in a substantially horizontal direction with respect to the support column part 23. The table part 25 functions as a worktable for performing ultrasound diagnosis.

As illustrated in FIG. 1, on the table part 25, a display 251 for displaying ultrasound image data, a console 252 for performing operations related to the display, and so forth are disposed. A support part 253 protrudes from the table part 25, and the display 251 is supported by the support part 253 from the back surface side.

A handle 254 is provided along the outer peripheral edge (outermost edge) of the table part 25. The handle 254 is for moving the cart 20 and the apparatus in a desired direction. The handle 254 is attached to, for example, the outer peripheral part of the holding frame 24 that holds the table part 25. The shape and so forth of the handle 254 are not limited to the illustrated example. In the illustrated example, the handle 254 is arranged so as to continuously surround the outer periphery of the table part 25. However, the handle 254 may not be continuous. The handle 254 may be partially provided on, for example, both sides or the back surface side of the ultrasound diagnostic apparatus 1.

As illustrated in FIG. 1 and so forth, a hook 255 is preferably provided on the lower surface side (back surface side) of the table part 25. The hook 255 is attached to, for example, the outer edge and so forth of the table part 25. Various cords and so forth can be suspended or hooked from the hook 255. The hook 255 may be attached to the table part 25 or may be provided on the holding frame 24, the handle 254, and so forth. The hook part 255 is not essential.

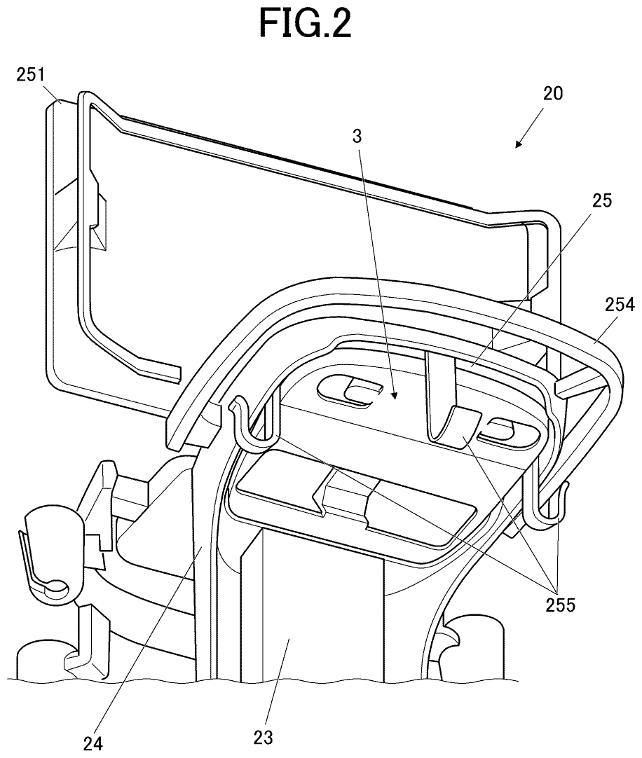

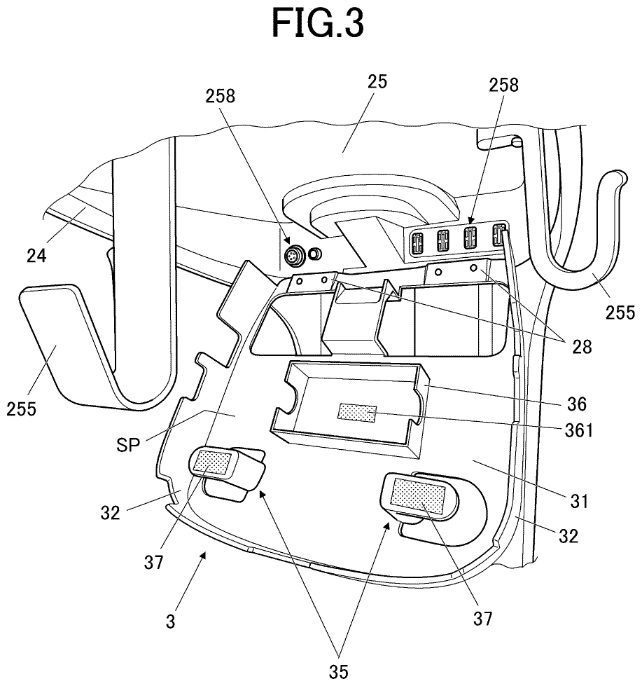

FIG. 2 and FIG. 3 are perspective views around the table part 25 of the ultrasound diagnostic apparatus 1 as viewed from diagonally below.

As illustrated in FIG. 2 and FIG. 3, a housing tray 3 is attached to the lower surface side (back surface side) of the table part 25. The housing tray 3 can be freely opened and closed with respect to the table part 25. FIG. 1 and FIG. 3 illustrate a state where the housing tray 3 is open with respect to the table part 25. FIG. 2 illustrates a state where the housing tray 3 is closed with respect to the table part 25.

The housing tray 3 is provided inside the outermost side edge of the table part 25. When the housing tray 3 is closed with respect to the table part 25, the entire upper side of the housing tray 3 is covered by the table part 25. Thus, dust and so forth is unlikely to enter the inside.

In the present embodiment, as shown in FIG. 1 and FIG. 2, the handle 254 for moving the apparatus is provided outside the outermost side edge of the table part 25. Thus, when the ultrasound diagnostic apparatus 1 hits a surrounding wall, a surrounding device, or the like while being moved, only the handle (grip) 254 outside the outermost side edge of the table part 25 hits the walls and so forth. That is, with the handle 254, the housing tray 3 provided on the inner side of the outermost edge of the table part 25 can be certainly prevented from hitting against surrounding things.

The handle 254 may not be attached to the holding frame 24. The handle 254 may be attached to, for example, the table part 25. However, the handle 254 is expected to have a cushioning effect when the ultrasound diagnostic apparatus 1 collides with an external device and so forth. From this viewpoint, it is preferable that the handle 254 is attached to the holding frame 24 rather than being directly attached to the table part 25. Thus, even when the handle 254 receives an impact due to collision with the surroundings, the impact to the table part 25 and the housing tray 3 provided on the lower surface side thereof can be more effectively mitigated.

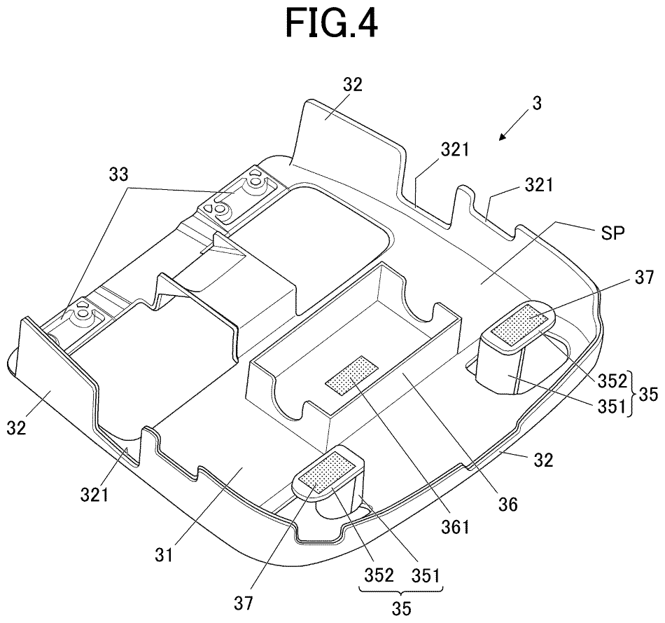

FIG. 4 is a perspective view illustrating the housing tray 3 alone detached from the table part 25.

As shown in FIG. 4, the housing tray 3 includes a bottom plate 31 and a side wall 32 rising from an outer peripheral part of the bottom plate 31. Part of the bottom plate 31 that is located above the grip part 12 of the apparatus main body 10 in the closed state is cut out. Thus, even when the holding frame 24 and the table part 25 are lowered, the housing tray 3 is prevented from interfering with the grip part 12 of the apparatus main body 10. It is not essential that the part of the bottom plate 31 is cut out. The housing tray 3 may not be cut out if the housing tray 3 is not configured to be lowered to a place at which the housing tray 3 interferes with the grip part 12. Instead of the cutout part, the bottom plate 31 may have a recess that is recessed inward so as to avoid the grip part 12.

One side of the housing tray 3 is rotatably attached to the table part 25 side, and the other side is swingable in a downward direction (indicated by an arrow in FIG. 1).

On the side of the housing tray 3 attached to the table part 25, hinge joint parts 33 (see FIG. 4) are provided. To the hinge joint parts 33, hinges 28 are attached. In the example illustrated in the embodiment, the housing tray 3 has the hinge joint parts 33 at positions on the side of the support column part 23 in the attached state. The hinges 28 are attached to the hinge joint parts 33 of the housing tray 3 and to the corresponding positions of the table part 25 side, so that the housing tray 3 is swingably hinged to the table part 25 (see FIG. 3).

In the embodiment illustrated in FIG. 3 and so forth, the hinges 28 for attaching the housing tray 3 to the table part 25 side are fixed to the inner side of the holding frame 24, which is provided so as to surround the table part 25.

The hinges 28 may not be attached to the inner side of the holding frame 24. One side of the housing tray 3 should be rotatably attached to the table part 25 side. The hinges 28 may be attached to, for example, the corresponding positions on the lower surface side of the table part 25.

As shown in FIG. 3 and FIG. 4, the housing tray 3 can house, in its internal space (housing space SP), at least an extra-length cable 51 of a peripheral device 50 that is related to the ultrasound diagnostic apparatus 1. On the inside of the housing tray 3, projection parts 35 on which the extra-length cable 51 can be wound are provided. In the illustrated example, the projecting parts 35 each include: a leg 351 that erects substantially vertically on the bottom plate 31 of the housing tray 3; and a catching arm 352 that bends substantially at a right angle from the leg part 351 and that is substantially parallel to the bottom plate 31.

The extra-length cable 51 is housed inside the housing tray 3, wound around the legs 351 of the projection parts 35, and caught by the catching arms 352. Thus, the extra-length cable 51 is held so as not to come off from the legs 351.

The shape and configuration of the part for winding the extra-length cable 51 are not limited to the illustrated example. The projection parts 35 are not essential for the housing tray 3.

The housing space SP may house the peripheral device 50 itself in addition to the extra-length cable 51. Examples of the peripheral device 50 to be mounted on the ultrasound diagnostic apparatus 1 include a printer (e.g., a monochrome printer), an electrocardiogram (ECG) recording device, and a barcode reader device.

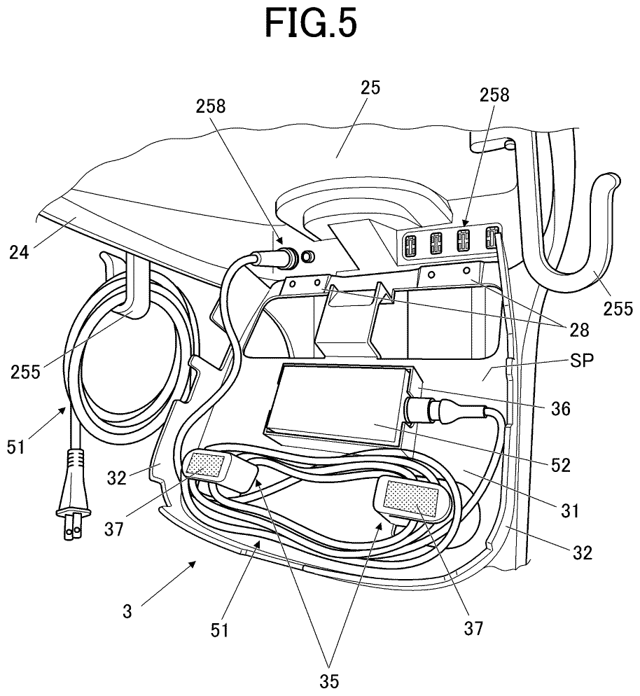

For example, in the example illustrated in FIG. 3 and FIG. 4, a device housing part 36 partitioned by a vertical wall is provided at the central part of the housing tray 3. The device housing part 36 has a size corresponding to, for example, the electrocardiogram recording device 52 so that the electrocardiogram recording device 52 can be placed therein (see FIG. 5).

When the housing tray 3 is closed with respect to the table part 25, the front surfaces (upper surfaces) of the catching arms 352 face the lower surface (back surface) of the table part 25 and are in contact with the lower surface (back surface) of the table part 25. On the surface (upper surface) of each of the catching arms 352, a tray-side locking means 37 is provided.

On the lower surface (back surface) of the table part 25, a table part-side locking means (not illustrated) is provided. The table part-side locking means is provided at the position corresponding to the tray-side locking means 37 when the housing tray 3 is in the closed state. The locking means 37 on the tray side and the locking means on the table part side are formed of, for example, hook-and-loop fasteners. The hook surface and the loop surface are appropriately determined for the locking means 37 on the tray side and the locking means on the table part side. When the housing tray 3 is in the closed state, the locking means 37 on the tray side and the locking means on the table part side are brought into contact with each other and engaged. Thus, the closed state of the housing tray 3 is maintained.

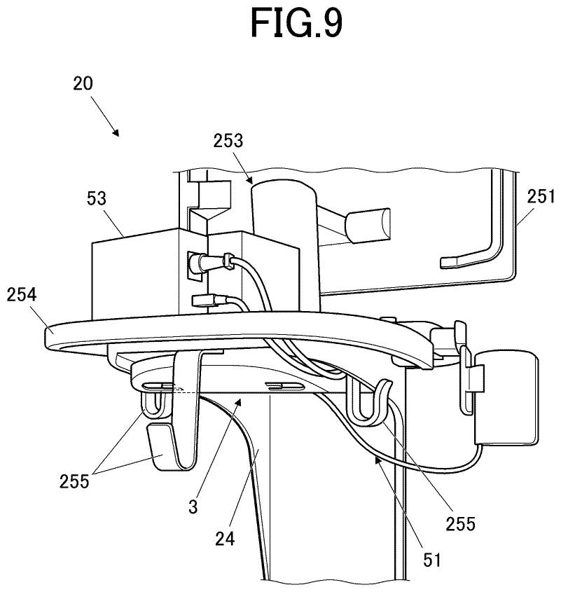

When the housing tray 3 is in the closed state, the upper side of the housing space SP is closed by the table part 25. Therefore, dust and so forth are unlikely to enter the housing space SP. When the housing tray 3 is in the closed state, the extra-length cable 51 and so forth housed in the housing space SP are not exposed to the outside (see FIG. 2 and FIG. 9). Therefore, the appearance of the ultrasound diagnostic apparatus 1 becomes simple, and the extra-length cable 51 and so forth are prevented from interfering with or being caught on the surroundings.

As shown in FIG. 3, in the embodiment, a connector 258 to which the peripheral device 50 is connected is provided in the housing space SP. The connector 258 is provided, for example, inside the holding frame 24. When the housing tray 3 is attached to the table part 25 and is in the closed state, the connector 258 is covered by the housing tray 3 and is housed in the housing space SP.

The connector 258 provided in the housing space SP is, for example, a power supply connector for supplying power to a printer and so forth, or a USB connector to which a USB terminal can be connected.

In the example shown in FIG. 3 and FIG. 5, as the connector section 258, a power supply connector is provided on the left side of the drawing, and four USB connectors are provided on the right side of the drawing.

The type of the connector 258 is appropriately determined. In the housing space SP, various connectors 258 may be provided, such as a connector for communication and a connector for connecting a video device. The number, type, arrangement, and so forth of the connectors 258 are not limited to the illustrated example.

The position of the connector 258 is not limited to the inside of the holding frame 24 as long as the connector 258 is at a position at which the connector part 258 is covered by the housing tray 3 and stored in the housing space SP when the housing tray 3 is in the closed state. For example, the connector 258 may be provided on the lower surface (back surface) of the table part 25.

As described above, the housing tray 3 is rotatable with respect to the table part 25 on the not-illustrated axis of the hinges 28. It is preferable that the housing tray 3 can rotate to the position at which the housing tray 3 is approximately at 90 degrees with respect to the table part 25 in the opened state. Thus, the extra-length cable 51 and so forth can be easily housed in the housing space SP and removed from the housing space SP.

As shown in FIG. 3 and so forth, a locking means 361, such as a hook-and-loop fastener, may also be provided in the device housing part 36. In such a case, the device to be housed in the device housing part 36, such as the electrocardiogram recording device 52, has a locking means corresponding to the locking means 361 provided in the device housing part 36.

Thus, even when the housing tray 3 is rotated to the position at substantially 90 degrees with respect to the table part 25, the device, such as the electrocardiogram recording apparatus 52, can be certainly prevented from falling from the housing tray 3.

Further, as shown in FIG. 4 and so forth, it is preferable that the side wall 32 has a cutout part 321 where appropriate so that the extra-length cable 51 housed in the housing space SP can be pulled out. The positions, the number, the sizes, and so forth of the cutout parts 321 are not limited to the illustrated example. In the embodiment, the extra-length cable 51 housed in the housing space SP can be appropriately pulled out from the cutout part 321 and can be connected to various external devices, such as the peripheral device 50 on the table part 25. It is preferable that as much of the extra-length cable 51 as possible is wound around the projection parts 35 so that the extra-length cable 51 pulled out outside of the housing tray 3 is short. Thus, the extra-length cable 51 can be prevented from becoming entangled or interfering with external devices and so forth outside the housing space SP.

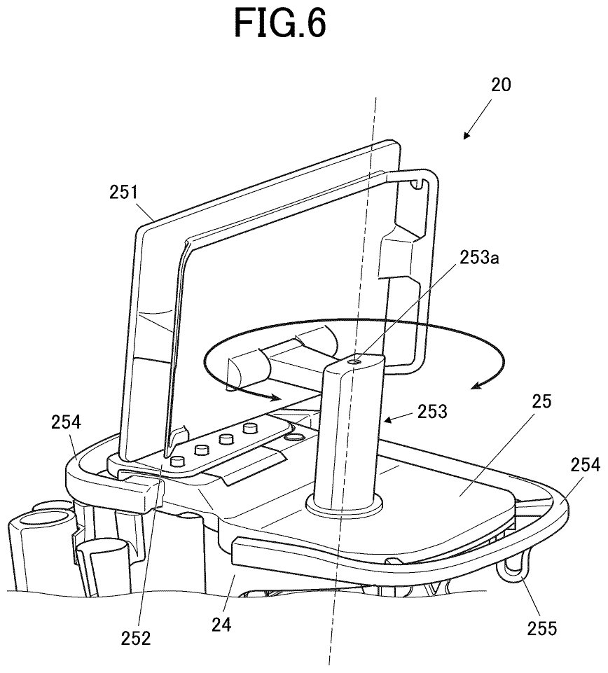

FIG. 6 is a main part perspective view of the table part 25 and its surroundings when viewed from the back of the cart 20.

As described above, the support part 253 supporting the display 251 from the back surface side erects on the table part 25. In FIG. 6, the axis passing through the center of the support part 253 is indicated by a chain line. In the illustrated example, the axis of the support part 253 slightly inclines backwards. The support part 253 is a rotation shaft of the display 251, and the display 251 is rotatable on the support part 253 in the direction of the arrow in FIG. 6. Thus, the orientation of the display 251 can be appropriately changed on the support part 253.

A screw hole 253a for attaching a camera is formed in the upper end surface of the support part 253. It is preferable that the screw hole 253a is closed by a cap (not shown) when the camera 7 is not attached.

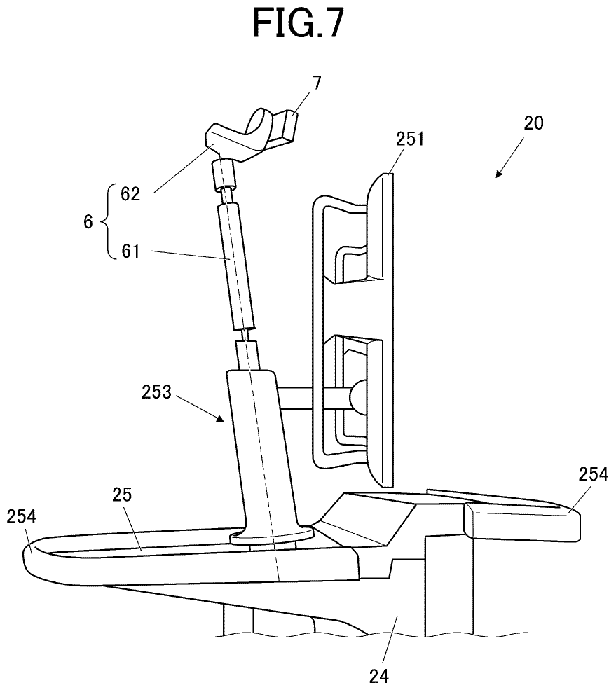

FIG. 7 illustrates a state in which a camera attachment unit 6 is attached to the camera-attachment screw hole 253a of the support part 253. In FIG. 7, the configuration on the table part 25, such as the console 252, is partially omitted.

The camera attachment unit 6 includes a leg 61 and a camera attachment part 62. The leg 61 has a shaft shape, and one end of the leg 61 has a screw (not illustrated) that can be screwed into the screw hole 253a. In the state the leg 61 is screwed in the screw hole 253a, the axial center of the leg 61 corresponds to the extended axial line of the support part 253. In FIG. 7, the axial center of the support part 253 and the leg 61 attached to the support part 253 is indicated by a dashed line. The length of the leg 61 in the axial direction may be adjustable.

The camera attachment part 62 is provided on the other end side (free end side) of the leg part 61. The camera attachment part 62 is, for example, a camera platform. It is preferable that the angle or the direction of the camera attachment part 62 with respect to the leg part 61 is adjustable. As illustrated in FIG. 7, a small camera 7 is attachable to the camera attachment part 62.

When the display 251 is rotated on the support part 253, the camera 7 is also rotated along with the display 251. Thus, it is not necessary to adjust the directions of the display 251 and the camera 7 individually, and the adjustment work can be simplified. In a case where the camera attachment part 62 is a camera platform, the direction of the camera 7 can be adjusted by adjusting the angle of the camera attachment part 62 regardless of the direction of the display 251.

The camera 7 can capture, for example, an image of a site of a subject (patient) on which the ultrasound probe (probe) is applied concurrently with ultrasound diagnosis. The image captured by the camera 7 may be a still image or a moving image. Audio information may be stored together with the image, for example. When the user examines the patient using the ultrasound diagnostic apparatus 1, a “body mark” may be added to an ultrasound image. The body mark indicates where and at what angle the ultrasound probe is placed on the subject (patient) to capture the image. In this regard, in the embodiment, the camera 7 captures an image of the site of the subject (patient) to which the ultrasound probe (probe) is applied. The image captured by the camera 7 may be superimposed on the ultrasound image during diagnosis, so that the superimposed image can serve as a substitute for the “body mark”. Thus, the user can check the examination state at a later diagnosis and can easily share information among medical professionals.

### [Operation of Ultrasound Diagnostic Apparatus]

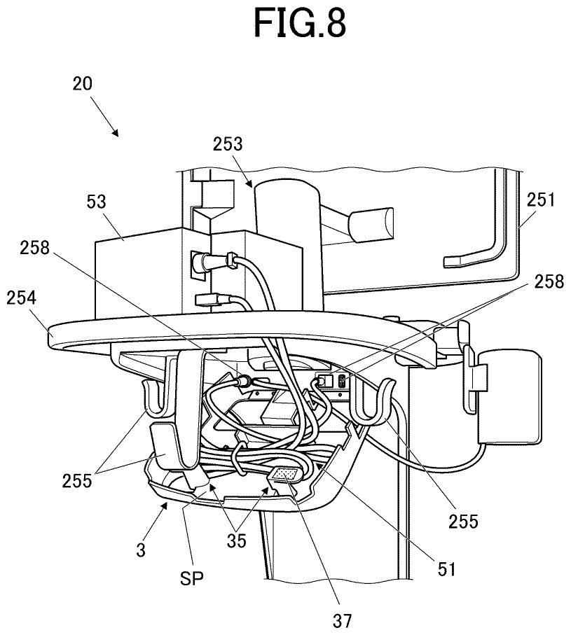

Next, the operation of the ultrasound diagnostic apparatus according to the present embodiment will be described with reference to FIG. 8 and FIG. 9.

In the present embodiment, the housing tray 3 is attached to the lower surface side of the table part 25 provided to the cart 20 of the ultrasound diagnostic apparatus 1 by using the hinges 28. Specifically, the hinges 28 are attached to the hinge joint parts 33 of the housing tray 3 and to the corresponding positions on the lower surface (rear surface) of the table part 25.

As illustrated in FIG. 4, a screw hole is formed in each hinge joint part 33. One wing of the hinge 28 is screwed to the hinge joint part 33 by a screw. The other wing of the hinge 4 is screwed to the corresponding position on the lower surface side of the table part 25. In the embodiment, the corresponding position on the lower surface side of the table part 25 is the inner side of the holding frame 24 at the support pillar part 23 side. In the state where the tube of one wing and the tube of the other wing are fitted to each other, a pin is inserted to the tube. Thus, the housing tray 3 is rotatably hinged to the table part 25.

Further, the tray-side locking means 37 is provided on the upper surface of the catching arm 352 of each projection part 35 provided in the housing tray 3. The table-top part-side locking means is provided on the lower surface of the table part 25 at the position corresponding to the tray-side locking means 37. That is, the hook surface of the hook-and-loop fastener is stick to the upper surface of the catching arm 352, and the loop surface of the hook-and-loop fastener is stuck to the corresponding position of the lower surface of the table part 25. Thus, the housing tray 3 is freely opened and closed with respect to the table part 25; and when the housing tray 3 is closed, the locking means 37 on the tray side is engaged with the locking means on the table part side, so that the closed state is maintained.

After the housing tray 3 is attached, the extra-length cable 51 and so forth are appropriately housed in the housing space SP in the housing tray 3. For example, in the example illustrated in FIG. 8, the peripheral device 50, such as a printer, is placed on the table part 25. The peripheral device 50 is connected to one end of a power cord or a USB cable. The other end of the power cable or the USB cable is connected to the connector 258 in the housing space SP. The extra-length cable 51, such as a power cable or a USB cable, of the peripheral device 50 is housed in the housing space SP.

Further, as shown in FIG. 5, the peripheral device 50, such as the electrocardiogram recording device 52, may be housed in the device housing part 36 in the housing tray 3, for example. In this case, the free end of the power cable opposite the end connected to the electrocardiogram recording apparatus 52 is connected to the connector part 258 in the housing space SP. The extra-length cable 51, such as the power cable, is appropriately wound around the projection part 35 and compactly housed in the housing space SP in the housing tray 3.

FIG. 9 is a main part perspective view of the housing tray 3 as viewed from diagonally behind, wherein the housing tray 3 is closed from the opened state of FIG. 8.

As shown in FIG. 9, when the housing tray 3 is in the closed state, the extra-length cable 51 and the peripheral devices 50, such as the electrocardiogram recording device 52, housed in the housing space SP in the housing tray 3 are not exposed to the outside.

The power cable and the USB cable can be pulled out by a necessary length from the cutout part 321 formed in the side wall 32 of the housing tray 3, the cutout part formed in the bottom plate 31, and so forth. Thus, even when the housing tray 3 is in the closed state, the power cable or the USB cable can be kept connected to the connector 258 in the housing space SP. Even when the ultrasound diagnostic apparatus 1 is moved between examination rooms in the state the power cable or the USB cable is connected, the power cable or the USB cable can be prevented from coming into contact with or being caught by an external device and so forth.

When the camera 7 is used instead of the “body mark”, the leg 61 of the camera attachment unit 6 is screwed to the screw hole 253a of the support part 253. The camera 7 is then attached to the camera attachment part 62 provided on the free end side of the leg part 61. When the display 251 is rotated on the support part 253, the camera 7 is also rotated along with the display 251, and thus the camera 7 can be easily directed toward the imaging target.

The camera 7 captures an image of the site and so forth of the subject (patient) to which the ultrasound probe (probe) is applied concurrently with the ultrasound diagnosis. Thus, it is possible to record where and at what angle the ultrasound probe (probe) is placed on the subject (patient) in capturing an ultrasound image. The records can be used to check the inspection state in a later diagnosis and to share information among medical professionals. The image captured by the camera 7 may be superimposed on the ultrasound image and displayed during diagnosis or may be saved separately.

### Effect

As described above, the ultrasound diagnostic apparatus 1 of the present embodiment includes: the cart 20; and the apparatus main body 10 to be mounted on the cart 20. The ultrasound diagnostic apparatus 1 is configured to be movable with the cart 20. The cart 20 includes the support pillar part 23 and the plate-shaped table part 25 that protrudes from the support pillar part 23 in a substantially horizontal direction. On the lower surface side of the table part 25, the housing tray 3 is provided so as to be freely open and closed with respect to the table part 25. The housing tray 3 can house at least the extra-length cable 51 of the peripheral device 50.

It is assumed that various peripheral devices 50 and multiple power cables and USB cables are connected to the ultrasound diagnostic apparatus 1. If the power cable and the USB cable are long with a margin (the extra-length cable 51), the extra-length cable 51 is likely to hit or be caught by structures inside and outside the room, such as various surrounding devices, a table, and a handrail when the ultrasound diagnostic apparatus 1 is moved. In this case, the cable may be broken, or the terminal part may receive an excessive force and may be damaged. In addition, various surrounding devices may be damaged.

In this regard, in the present embodiment, the ultrasound diagnostic apparatus 1 includes the housing tray 3 capable of housing the extra-length cable 51. Such a housing tray 3 can prevent the extra-length cable 51 from being caught. Thus, when the ultrasound diagnostic apparatus 1 is movable, it is possible to reduce the risk of damaging the peripheral device 50 and its cables and eventually reduce the risk of damaging other devices and various kinds of equipment in the facility due to the movement.

Further, in the present embodiment, the housing space SP, which is formed by the housing tray 3, has therein a space for housing the peripheral device 50. That is, in the embodiment, the device housing part 36 is provided in the housing space SP in the housing tray 3. The housing tray 3 can house not only the extra-length cable 51 but also the peripheral device 50, such as the electrocardiogram recording device 52.

Thus, various peripheral devices 50 to be used together with the ultrasound diagnostic apparatus 1 can be compactly moved together with the ultrasound diagnostic apparatus 1.

Further, in the housing space SP formed by the housing tray 3 in the present embodiment, the connector 258 is provided to which the peripheral device 50 is to be connected.

By providing the connector 258, the peripheral device 50 can be easily connected and used. Since the inside of the housing space SP is invisible from the outside when the housing tray 3 is in the closed state, the appearance can be simplified.

Further, when the housing tray 3 is in the closed state, dust and so forth are unlikely to enter the housing space SP and do not contact surrounding devices. Thus, the connector 258 can be kept clean and safe.

Further, in the present embodiment, one end of the housing tray 3 is rotatably attached to the table part 25; and when the housing tray 3 is in the open state, the other end of the housing tray 3 is swingable to a position where the housing tray 3 is approximately at 90 degrees with respect to the table part 25.

Thus, the extra-length cable 51 and so forth can be easily taken in and out of the housing space SP in the housing tray 3, and can be easily handled. Further, since the housing tray 3 is widely opened, the devices and so forth housed in the housing space SP can be easily checked.

Further, in the embodiment, the housing tray 3 is provided inside the outermost edge of the table part 25.

Thus, the entire upper side of the housing tray 3 provided on the lower surface side of the table part 25 is covered by the table part 25, so that dust and so forth are prevented from entering the inside. Further, since the housing tray 3 does not protrude outward than the table part 25, the housing tray 3 is prevented from directly contacting surrounding devices, equipment, and so forth.

Further, in the present embodiment, the handle 254 for moving the apparatus is provided at the outside of the outermost edge of the table part 25.

When the ultrasound diagnostic apparatus 1 hits against surrounding equipment, facilities, and so forth, the handle 254 is firstly in contact with these surroundings. Thus, the table part 25 and the housing tray 3 provided on the lower surface of the table part 25 can be prevented from hitting against the surroundings. Further, if the handle 254 is made of a material having a cushioning property, such as resin, a buffering effect can also be expected. By providing the handle 254 at the outside of the outermost side edge of the table part 25, it is possible to alleviate the impact on the apparatus main body 10 mounted on the cart 20 and the peripheral device 50 placed on the table part 25.

Further, in the present embodiment, the housing tray 3 internally includes the projection part 35 around which the extra-length cable 51 can be wound.

Thus, the long extra-length cable 51 can be compactly housed in the housing tray 3.

Furthermore, in the present embodiment, the housing tray 3 includes the tray-side locking means 37; and the table part 25 includes the table part-side locking means (not illustrated) at the position corresponding to the tray-side locking means 37 when the housing tray 3 is in the closed state. The tray-side locking means 37 is engaged with the table part-side locking means, so that the closed state of the housing tray 3 is maintained.

Thus, the housing tray 3 is prevented from being erroneously opened, and the extra-length cable 51 and so forth housed therein are prevented from popping out.

### Others

Although the embodiment of the present invention has been described above, the embodiment is not intended to limit the present invention and can be variously modified without departing from the scope of the present invention.

For example, in the above embodiment, the tray-side locking means 37 and the table-top-part side locking means are the hook-and-loop fastener as an example. However, the locking means 37 on the tray side and the locking means on the table part side are not limited to the hook-and-loop fastener as long as they can maintain the closed state of the housing tray 3. For example, magnets that attract each other may be provided on the housing tray 3 and the table part 25; or a hook may be used to lock the housing tray 3 to the table part 25.

Further, the articles housed in the housing space SP of the housing tray 3 are not limited to the extra-length cable 51 or the peripheral device 50, such as the electrocardiogram recording device 52.

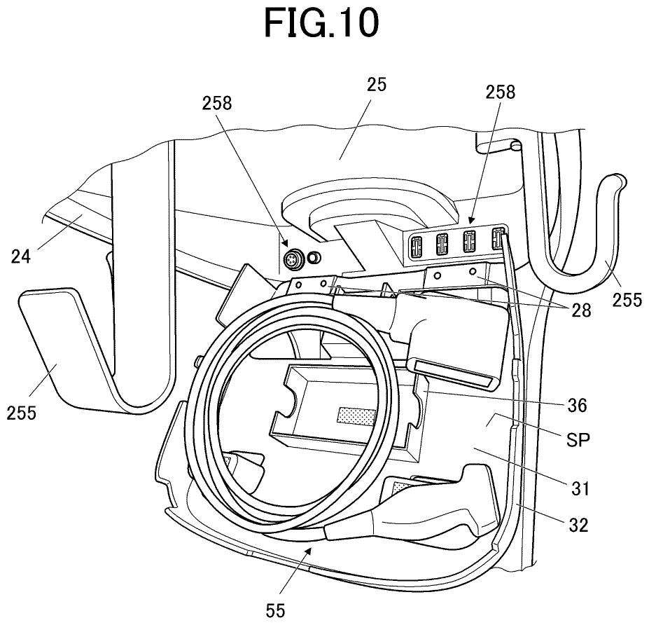

For example, as shown in FIG. 10, an extra probe 55 may be housed in the housing space shown in FIG. 3.

An ultrasound probe (probe) needs to transmit an ultrasound wave toward a region to be diagnosed and accurately receive a reflected wave from an organ or a tissue. Therefore, there is a case where ultrasound probes most suitable for the respective diagnosis target regions are prepared and used as necessary. For example, in diagnosing multiple sites, it is expected that multiple types of ultrasound probes are housed in the housing tray 3 and carried together with the ultrasound diagnostic apparatus 1. The extra probe 55 is not limited to a different type. A spare probe may be prepared in case of malfunction of the probe.

Further, multiple types of housing trays 3 having different housing spaces SP may be prepared; and the housing tray 3 having an optimal configuration may be attached, depending on how the ultrasound diagnostic apparatus 1 is used in the facility.

For example, the housing tray 3 may not necessarily have the device housing part 36 corresponding to the peripheral device 50, such as the electrocardiogram recording apparatus 52. When the extra probe 55 needs to be housed in the housing space SP in the housing tray 3 as described above, the extra probe 55 may be easily housed when there is no device housing part 36. In such a case, the housing tray 3 may not have the device housing part 36 and so forth so that various devices can be freely housed in the housing tray 3.

Although the embodiments of the present invention have been described above, the scope of the present invention is not limited to the above embodiments. The specific configuration of the ultrasound diagnostic apparatus 1 shown in the above embodiments, the positional relationship and operations of the components, and so forth can be appropriately changed without departing from the scope of the present invention. The scope of the present invention includes the scope of the invention described in the scope of the claims and the scope of equivalents thereof. The scope of the present invention should be interpreted by terms of the appended claims.

1. An ultrasound diagnostic apparatus comprising:
a cart; and
an apparatus main body, wherein:
the ultrasound diagnostic apparatus is configured to be movable in a state where the apparatus main body is mounted on the cart,
the cart includes a support pillar part and a plate-shaped table part that protrudes from the support pillar part in a substantially horizontal direction, and
on a lower surface side of the table part, a housing tray is attached so as to be freely opened and closed with respect to the table part, the housing tray being capable of housing at least an extra-length cable of a peripheral device.

2. The ultrasound diagnostic apparatus according to claim 1, wherein the housing tray forms a housing space in which a space for housing the peripheral device is secured.

3. The ultrasound diagnostic apparatus according to claim 1, wherein in a housing space formed by the housing tray, a connector to which the peripheral device is connectable is provided.

4. The ultrasound diagnostic apparatus according to claim 1, wherein:
one end of the housing tray is rotatably attached to a table part side, and another end of the housing tray is swingable in a downward direction, and
when the housing tray is opened, state, the housing tray swings to a position where the housing tray is substantially at 90 degrees to the table part.

5. The ultrasound diagnostic apparatus according to claim 1, wherein the housing tray is provided inside an outermost edge of the table part.

6. The ultrasound diagnostic apparatus according to claim 1, wherein a handle for moving the apparatus is provided outside an outermost edge of the table part.

7. The ultrasound diagnostic apparatus according to claim 1, wherein in the housing tray, a projection part on which the extra-length cable can be wound is provided.

8. The ultrasound diagnostic apparatus according to claim 1, wherein:
the housing tray includes a tray-side locking means,
the table part includes a table part-side locking means at a position corresponding to the tray-side locking means of the housing tray in a closed state, and
the closed state of the housing tray is maintained by the tray-side locking means and the table part-side locking means engaging with each other.
